(19) United States
(12) Patent Application Publication
(10) Pub. No.: US 2006/0177929 A1
(43) Pub. Date: Aug. 10, 2006

(54) Regulation of self-renewal in stem cells

(76) Inventor: Christopher A. Klug, Birmingham, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 10/550,657

(22) PCT Filed: Mar. 22, 2004
(86) PCT No.: PCT/US04/08607

§ 371 Date: Sep. 26, 2005

Related U.S. Application Data
(60) Provisional application No. 60/457,152, filed on Mar. 24, 2003.

(51) Int. Cl. C12N 5/08

(52) U.S. Cl. 435/372

(57) ABSTRACT

Disclosed are methods for regulating the self-renewal capacity of stem cells such as, but not limited to, primitive hematopoietic stem cells (HSCs) (both mouse and human) by modulating the function and/or activity of target factors that are controlled or altered in their regulation by AML1-ETO expression in stem cells such as HSC. Target factors include, but are not limited to, AML1, C/EBP alpha, and/or PU.1 either individually, or in combinations, Altering the function and/or activity of such target factors in HSC leads to inhibition of HSC differentiation and stimulation of HSC self-renewal capacity. Modulation of target factor activity may be achieved at the level of synthesis of these target factors, interaction with cellular factors required for basal activity or enhanced activity, such as cofactors, interactions with their DNA binding motifs, or by targeted degradation or inhibition of their mRNAs.

This application claims priority to and the benefit of U.S. provisional patent application No. 60/457,152 filed Mar. 22, 2003.

### FIELD OF THE DISCLOSURE

The present disclosure is related to methods for expansion of stem cells. Specifically, the present disclosure is directed to methods for expansion of stem cells by reducing the ability of stem cells to undergo cellular differentiation while preserving their ability to undergo self-renewal.

### BACKGROUND

Stem cells may be defined as cells that can divide to produce other stem cells (self-renewal) as well as cells that can differentiate (under appropriate cellular signals) along multiple differentiation pathways. Stem cells play a critical role in physiology, allowing the organism to undergo development, and further, allowing the organism to maintain that development throughout life in tissues, like the blood system. Stem cells can be found at all stages of development. For example, in humans embryonic stem cells are formed at the blastocyst stage shortly after egg fertilization by the sperm. Embryonic stem cells are totipotent, meaning they can produce progeny capable of developing into any cell type in the body. Other types of stem cells, often referred to as adult stem cells, are present in the various tissues throughout the body (such as the blood, brain and muscle). Adult stem cells are thought to be pluripotent, meaning the stem cells are limited to producing progeny capable of developing into cell types only from the tissue of origin of the adult stem cell (for example, hematopoietic stem cells giving rise to T-cells and hematopoietic blood cell types, but not neural cells). However, recent research suggests that adult stem cells may have more plasticity than originally believed, and may be able to give rise to a wider variety of cell types.

The blood and immune systems of adult mammals are generated and maintained throughout life by a rare population of self-renewing hematopoietic stem cells (HSCs) active in adult bone marrow. HSCs may be operationally defined as pluripotent cells that can self-renew to reconstitute the hematopoietic system following transplantation into lethally-irradiated recipient mice. (Morrison et al., 1994; Weissman, 2000). Long-term self-renewing HSC (LT-HSC) generate donor-derived blood cells in a recipient animal for its remaining life-span while short-term self-renewing HSC (ST-HSC) only give rise to blood cells for 8-12 weeks, after which time donor-derived hematopoiesis ceases (Morrison et al., 1994). Both LT-HSC and ST-HSC retain their ability to generate all of the blood cell types (i.e., they are pluripotent) and can be purified as distinct populations using the fluorescence-activated cell sorter (FACS). A single LT-HSC can regenerate the entire hematopoietic system of a lethally irradiated mouse for its remaining life span, which illustrates the extensive self-renewal capacity of this cell population.

The progeny of stem cells undergo various differentiation stages until they reach a state of terminal differentiation (developmental maturity). The differentiation stages are regulated by intricate signaling cascades and unique combinations of transcription factors, which translate specific signaling information into specific patterns of gene expression. The factors that are responsible for these processes are not fully understood for most cell types. By intervening in the process that regulates stem cell differentiation, it may be possible to grow HSCs in vitro and/or manipulate the differentiation process in vivo.

Expansion of HSCs in vitro would have enormous clinical potential. To date, however, there has been only a limited success in expanding HSCs in vitro. Most attempts have centered on using a variety of cytokine incubation conditions as measured by competitive repopulation assays with expanded cells (Miller and Eaves, 1997). The inability to expand HSCs is most likely due to the limitations of the existing approaches and to our limited knowledge of basic HSC biology. The success of in vitro expansion of other stem cell types, including embryonic stem cells (Evans and Kaufman, 1981; Martin, 1981) and neuronal stem cells (Reynolds and Weiss, 1992), and the observation that HSCs expand in vivo upon transplantation into irradiated mice (Dick et al., 1985; Lemischka et al., 1986) suggest that better understanding of HSC biology at the molecular level may lead to development of protocols for HSC expansion.

An additional approach to understanding the self-renewal process in HSCs and the molecular controls that regulate this process is to use animal models to study HSC expansion. There have been relatively few examples where HSC numbers in vivo have been shown to extensively expand beyond the normal limitations imposed by the genetic control of HSC pool size (de Haan and Van Zant, 1997; Muller-Sieburg and Riblet, 1996). Two such examples involve the homeobox-containing genes, Hoxb4 and Hoxa9 (Sauvageau et al., 1995; Thorsteinsdottir et al., 2002). In the latter case, HOXA9 expressed in hematopoietic cells using a retroviral vector led to a 15-fold increase in transplantable long-term repopulating cells, although animals developed a myeloproliferative disorder in this context. Mice reconstituted with cells expressing HOXB4 showed a 50-fold increase in stem cell numbers as measured in transplantation assays. This increase was associated with apparently more rapid self-renewal in the early stages of reconstitution, with the absolute number of stem cells in long-term reconstituted animals still being sensitive to homeostatic control of stem cell pool size in vivo. Sonic hedgehog (Bhardwaj et al., 2001) and the Notch 1 (Varnum-Finney et al., 2000) signaling pathways have also been implicated in the regulation of HSC self-renewal.

The present disclosure provides a method for expanding stem cells by inhibiting the differentiation potential of the stem cells without inhibiting the ability of the stem cells to undergo self-renewal. The method is demonstrated in one embodiment by using a novel animal model of stem cell expansion created by introducing the AML1-ETO gene into HSCs and introducing these HSCs into lethally irradiated mice (de Guzman et al., 2002). In this model of stem cell expansion, hematopoietic stem cells that express AML1-ETO can expand at least 100-fold in vivo. This expansion was not associated with an increased HSC proliferation rate but was rather marked by a reduced ability of HSC to differentiate in the presence of AML1-ETO. The ability of AML1-ETO to enhance self-renewal of HSC in vitro has also been documented by in vivo long-term reconstitution experiments with in vitro-expanded cells. Using the teachings of the present disclosure, methods are provided for expanding a population of stem cell by modulating a target factor involved in the cellular biochemical pathways regulating cellular differentiation and self-renewal in the stem cells. In one embodiment, the target factor is involved in the biochemical pathways regulated by the AML1-ETO gene product. Therefore, the present disclosure provides a means to control stem cell expansion in vitro.

### BRIEF DESCRIPTION OF THE DRAWINGS

FIGS. 1A-D illustrate the retroviral transduction of murine hematopoietic stem cells.

FIG. 1A shows a schematic diagram of one embodiment of a MSCV retroviral constructs used in the present disclosure, with the MSCV AML1-ETO IRES GFP shown on top and the control MSCV IRES GFP shown on the bottom.

FIG. 1B illustrates the results of the gating procedure used for sorting the HSC phenotype, c-kit+Sca-1+Lin−, where Lin represents a cocktail of antibodies to the mature blood cell antigens Mac-1, Gr-1, Ter119, B220, CD3, CD4, CD5 and CD8.

FIG. 1C illustrates flow cytometric analysis of HSC after 24-hour retroviral transduction. Approximately 300, Ly-5.2+ HSCs from control or AML1-ETO transductions were transplanted with a radioprotective dose of 2×10⁵ Ly-5.1+ whole bone marrow cells into each Ly-5.1+ recipient animal.

FIG. 1D illustrates Western blot analysis of GFP+ (lane 1) or GFP− (lane 2) myeloid scatter-gated cells FACS-sorted from the bone marrow of an 8-week post-transplant AML1-ETO animal probed with a polyclonal anti-AML1 antibody with lane 1 indicating AML1-ETO expression at the 8-week time point.

FIGS. 2A-C illustrate abnormal myelopoiesis and decreased B lymphopoiesis in AML1-ETO/GFP+ peripheral blood cells.

FIG. 2A illustrates flow cytometric analysis of peripheral blood cells from animals at 2.5 months post-transplantation stained with an antibody to the Ly-5.2 donor marker.

FIG. 2B illustrates analysis of peripheral blood cells gated to select GFP⁻ or GFP⁺ populations for simultaneous Mac-1 and Gr-1. FACS plots are representative of all co-cultured whole bone marrow transplants of control GFP (n=21) and AML1-ETO-expressing (n=26) mice and all purified HSC transplants of control GFP (n=5) and AML1-ETO (n=3) mice.

FIG. 2C illustrates analysis of peripheral blood cells gated to select GFP⁻ or GFP⁺ populations for B220 expression. FACS plots are representative of all co-cultured whole bone marrow transplants of control GFP (n=21) and AML1-ETO-expressing (n=26) mice and all purified HSC transplants of control GFP (n=5) and AML1-ETO (n=3) mice.

FIGS. 3A-D illustrate abnormal myelopoiesis in AML1-ETO-expressing bone marrow cells.

FIG. 3A illustrates flow cytometric analysis of bone marrow from a 10-month post-transplant AML1-ETO mouse. Bone marrow cells were gated to select (1) GFP− and (2) AML1-ETO/GFP+ bone marrow cells and analyzed for expression of Mac-1 and Gr-1. The data are representative of all AML1-ETO transplanted animals between 2-10 months post-transplant. The Mac-1/Gr-1 profile in (1) is identical to what is seen in bone marrow from control GFP animals.

FIG. 3B shows a Wright-Giemsa stained cytospin preparation of AML1-ETO/GFP+, Mac-1^{HI}Gr-1^{int} cells gated as shown in FIG. 3A (100× magnification). Arrows indicate (a) banded neutrophil and (b) metamyelocyte.

FIG. 3C illustrates graded levels of AML1-ETO expression produce distinct Mac-1/Gr-1 phenotypes in bone marrow.

FIG. 3D illustrates Northern blot analysis of RNA isolated from GFP− and AML1-ETO/GFP+ bone marrow cells from a 3-month post-transplant AML1-ETO animal. The blot was probed with a 3′ fragment of the C/EBP alpha cDNA and a GAPDH probe. Quantitation of transcript levels was done on a phosphoimager.

FIGS. 4A-D demonstrate an increase in myeloid colony-forming cells in AML1-ETO animals.

FIG. 4A illustrates myeloid-scatter gating of cells into GFP− or AML1-ETO/GFP+ populations from a 10-month-old AML1-ETO mouse.

FIG. 4B shows the colonies obtained when 1000 cells from each population described in FIG. 4A were plated in triplicate into M3434 methylcellulose media supplemented with 0.5 ng/ml GM-CSF. Three independent AML1-ETO animals at 2 and 10 months post-transplant were used in the analysis. Colonies were enumerated and characterized 10 days after plating.

FIG. 4C illustrates representative FACS plots of individual methylcellulose colonies stained with Mac-1 and Gr-1. Two plots are shown for each sample.

FIG. 4D illustrates cytospin preparations of GFP− and AML1-ETO/GFP+ colonies stained with Wright-Giemsa. Arrows indicate mature, segmented neutrophils among the GFP− cells that were not seen in any AML1-ETO-expressing colonies.

FIGS. 5A and B demonstrate expansion of hematopoietic stem cells in AML1-ETO mice.

FIG. 5A illustrates a HSC analysis from a 10-month post-transplant AML1-ETO mouse. Bone marrow cells were stained with c-kit, lineage marker antibodies (see Methods), Sca-1, and the Ly-5.2 donor marker. The percentage of cells in individual gated populations is indicated.

FIG. 5B illustrates the results of the procedure described in FIG. 5A but using HSC obtained from a control GFP animal.

FIGS. 6A and B demonstrate delayed differentiation in AML1-ETO-expressing stem cells.

FIG. 6A illustrates the percentage of AML1-ETO-expressing (GFP+) cells in the stem cell population and in whole bone marrow at 2 months (n=3) post-reconstitution.

FIG. 6B illustrates the percentage of AML1-ETO-expressing (GFP+) cells in the stem cell population and in whole bone marrow at 10 months (n=3) post-reconstitution. The ratio of GFP+ cells in the stem cell compartment and in the bone marrow of control GFP animals was similar to the ratio seen in older AML1-ETO animals.

FIG. 7 demonstrates that AML1-ETO expression in stem cells is required for maintenance of abnormal myelopoiesis.

Bone marrow from one primary recipient AML1-ETO animal was serially transplanted at a dose of 4×10⁶ cells into each of four, lethally-irradiated secondary mice. Flow cytometric analysis of HSC in 1 out of 4 secondary animals is shown at 5 weeks post-transplant. All secondary transplant animals received 114,000 AML1-ETO-expressing myeloid cells along with approximately 600 AML1-ETO/GFP+ HSC in the bone marrow inoculums (WBM, whole bone marrow).

FIGS. 8A and B show that AML1-ETO directly influences self-renewal of HSC.

FIG. 8A shows reconstitution of B-cell lineages four months post-transplant where animals were reconstituted with HSC expressing AML1-ETO-ER in the presence of 4-HT.

FIG. 8B shows reconstitution of T-cell lineages four months post-transplant where animals were reconstituted with HSC expressing AML1-ETO-ER in the presence of 4-HT.

### SUMMARY

Using the teachings of the present disclosure, methods are provided for expanding a population of stem cell by modulating a target factor involved in the cellular biochemical pathways regulating cellular differentiation and self-renewal in the stem cells. In one embodiment, the self-renewal capacity of the stem cells is regulated by inhibiting the ability of the stem cells to differentiate while preserving the ability of the stem cells to undergo self-renewal. In one embodiment, the target factor is involved in the biochemical pathways regulated by the AML1-ETO gene product. Therefore, the present disclosure provides a means to control stem cell expansion in vitro.

In one embodiment, the stem cells are HSCs. Therefore, the present disclosure is also directed to methods for expanding a population of HSCs by regulating the self-renewal capacity of primitive HSCs (both mouse and human) by regulating the activity of target factors that are being influenced, either directly or indirectly, by AML1-ETO expression in HSC. In one embodiment, the self-renewal capacity of the HSCs is regulated by inhibiting the ability of the HSCs to differentiate while preserving the ability of the HSCs to undergo self-renewal. The inhibition of differentiation may be total or partial. In one embodiment, the target factors are proteins. Target factors to be regulated to achieve HSC expansion include, but are not limited to, AML1, C/EBP alpha, and/or PU.1 either individually, or in combinations. It has been demonstrated that AML1-ETO inhibits the function of the wild-type AML1 protein and also inhibits the expression and function of C/EBP alpha and PU.1. However, this inhibition of transcription factor activity has not been shown to lead to regulation of HSC differentiation and/or expansion. The present disclosure demonstrates that AML1-ETO expression in HSC leads to inhibition of HSC differentiation and stimulation of HSC self-renewal capacity. Without being limited to other explanations, this effect of AML1-ETO, may be due to modulation of the function of target factors, such as, but not limited to, AML1, C/EBP alpha, and/or PU.1 in HSC. Modulation of function may include inhibition of the function of target factors, stimulation of the function of the function of target factors or translocation of the activity of the target factors.

These target factors (and others) may be responsible for inducing the first differentiation event within HSC. By modulating the activity of the target factors, such as, but not limited to, AML1, C/EBP alpha and/or PU.1, the differentiation potential of HSCs/precursor cells is reduced without destroying the ability of HSC to self-renew. Modulation of target factor activity may lead to the modulation of other factors in the HSC. The target HSC population to be used for expansion may be isolated from a substantially purified or partially purified population of HSCs/precursor cells from any tissue that may harbor adult HSCs or other stem cells, including, but not limited to, bone marrow, peripheral blood, muscle, skin, adipose tissue, or tissue derived from the nervous system.

Modulation of target factor activity, such as, but not limited to, AML1, C/EBP alpha and/or PU.1, may be achieved in many different ways. Modulation may occur at the level of synthesis of these factors, interaction with cellular factors required for basal activity or enhanced activity, such as cofactors (AML1/C/EBP alpha/PU.1 interactions), interactions with their DNA binding motifs (transcription factor-DNA interactions), by altering the degradation rate or transcription rate of target factor mRNA, such as by targeted degradation, or using methods like small interfering RNAs (RNAi) (Elbashir et al., 2001) or peptide nucleic acids (Ray and Norden, 2000). These modulations may be direct or indirect.

The present disclosure shows that AML1-ETO inhibits HSC differentiation while not altering the ability of HSC to undergo cell divisions that lead to self-renewal in vivo. The in vitro-expanded population of HSC/precursor cells may be used to replace or supplement the cell population of a subject to which the expanded population of precursor cells are administered. For example, the expanded population of stem cells may be administered to a subject to replace the hematopoietic system after extensive chemotherapy or radiation for numerous types of cancer. Alternatively, these cells may be used as a source of adult stem cells that can be used to generate and replace other cell types found in other tissues like the liver, pancreas, skin, or the nervous system. They may also be used as a means to allow gene therapy treatments with expanded, gene-modified cells, and to replace diseased or degenerating cell populations in the subject.

It is an object of the disclosure to inhibit the differentiation and/or promote the self-renewal of stem cells such as HSCs by regulating the activity of target factors that control HSC differentiation and/or self-renewal. The target factors to be specifically targeted are those that are being mis-regulated as a consequence of AML1-ETO expression. It is an additional object to provide regulation of factors that control stem cells differentiation and/or self-renewal in a reversible manner. Additionally, it is an object of the disclosure to provide such reversible regulation by regulating the expression or activity of such target factors. It is yet another object of the disclosure to provide a method for the expansion of stem cells, such as HSC cells by inhibiting the differentiation and/or promoting the self-renewal of such cells. Finally, it is a further object of the disclosure to produce stem cells, such as HSC cells, for therapeutic purposes for use in subjects in need of such treatment.

### DETAILED DESCRIPTION

As used in the present disclosure, “stem cells” means a population of self-renewing, undifferentiated cells that can be found in a number of mammalian tissues and organs that serve as a reservoir to replace more terminally differentiated cells that are lost in those tissues or organs. Stem cells include “hematopoietic stem cells” (HSCs). HSCs means the rare population of cells that can both self-renew and differentiate into all of the cell types found in the mammalian blood and immune systems.

AML1, C/EBP Alpha and PU.1

The present disclosure is directed to methods for regulating the self-renewal capacity and/or differentiation capacity of stem cells by regulating the activity of target factors that are misregulated in HSC by AML1-ETO. In one embodiment, the stem cells are HSC. In one embodiment, the self-renewal capacity of the HSCs is regulated by inhibiting the ability of the HSCs to differentiate while preserving the ability of the HSCs to undergo self-renewal. The inhibition of differentiation may be total or partial. Such factors may include, but are not limited to, AML1, C/EBP alpha, and/or PU.1, which are critical in the differentiation/self-renewal potential of HSCs. Observations related to the increased self-renewal of HSC in vivo in the presence of AML1-ETO have only been documented by studies described in de Guzman et al. (2002). Other studies have shown that AML1-ETO can increase the number of human, primitive colony-forming cells in vitro (the Applciant has also shown this with mouse cells) but there is no colony-forming cell assay that unequivocally defines a long-term repopulating HSC (Mulloy et al., 2002). The instant disclosure demonstrates for the first time that HSC numbers are increased by AML1-ETO both with respect to the HSC cell-surface phenotype in vivo and with respect to ex vivo expansion of long-term repopulating cells. The in vivo long-term repopulation assay is the only unequivocal means of establishing and quantifying stem cell expansion. Pathways being regulated by AML1-ETO in HSC may be involved in the differentiation pathway of other hematopoietic precursor cells, including myeloid progenitor cells, leading to a reduction in the ability of these precursor cells to undergo the normal myeloid differentiation program. The reduced ability to differentiate may depend on the level of inhibition of target factors that are misregulated by AML1-ETO activity, such as, but not limited to, AML1, C/EBP alpha, PU.1.

AML1 (also known as Runx1) is a transcription factor with significant homology to the Drosophila segmentation gene, Runt (Miyoshi et al., 1991; Erickson et al., 1992). It binds the enhancer core target sequence, TGT/cGGT, in association with a non-DNA-binding subunit, CBFβ (Wang et al., 1993; Ogawa et al., 1993; Meyers et al., 1993; Bravo et al., 2001). Both proteins (together referred to as core binding factor or CBF) interact through the DNA-binding, the Runt homology domain of AML1. Null mutations in either CBF subunit in mice resulted in embryonic lethality that was associated with intra-cranial hemorrhaging and a complete absence of definitive hematopoiesis (Okuda et al., 1996; Wang et al., 1996a; Wang et al., 1996b; Sasaki et al., 1996). The complete absence of hematopoietic cells in AML1 knockout animals indicates that AML1 is essential for the formation of differentiated blood cells from HSCs (Okuda et al., 1996).

Mutations in the AML1 gene, including chromosomal translocations, represent one of the most common genetic abnormalities observed in leukemia. The t(8;21)(q22;q22) translocation, which fuses the ETO gene on human chromosome 8 with the AML1 gene on chromosome 21, is seen in approximately 12-15% of acute myelogenous leukemia (AML) cases, and in about 40% of AML with a French-American-British classified M2 phenotype (reviewed in Nucifera and Rowley, 1995; Downing, 1999). The t(8;21) translocation fuses the N-terminal 177 amino acids of AML1, which includes the Runt homology domain that binds DNA and interacts with CBFβ, in frame with amino acids 30-604 of ETO. The fusion protein deletes the C-terminal activation domain of AML1. The ETO gene is homologous to the Drosophila gene, nervy, and can associate with transcriptional co-repressor complexes that include mSin3, histone deacetylates (HDACs), and nuclear hormone co-repressors, which are involved in transcriptional repression (Lutterbach et al., 1998). Gene knock-in experiments in mice have shown that AML1-ETO acts in a dominant-repressive manner to block AML1-dependent transcription (Yergeau et al., 1997; Okuda et al., 1998). Animals heterozygous for an AML1-ETO knock-in allele displayed a similar phenotype to AML1 or CBFβ knock-out mice in that they died early in embryonic life (e13.5) and exhibited intra-cranial bleeding and a block in definitive hematopoiesis. One important difference between the knock-out and knock-in phenotypes was the presence of dysplastic hematopoietic progenitor cells within the fetal livers of the knock-in mice that could readily be established as immortalized cell lines in vitro (Okuda et al., 1998). The similarity of the AML1 knockout results with the AML1-ETO knock-in mice indicates that AML1 is an important target protein for HSC function and may be a primary effector protein for HSC self-renewal, since there are no definitive hematopoietic cells in the absence of AML1. These results may also indicate that AML1 is important for HSC differentiation into the various blood cell lineages.

The consequence of AML1-ETO expression on myeloid lineage development has been explored using transformed myeloid cell lines that retain some capacity to terminally differentiate. Expression of AML1-ETO in the myeloid cell line 32D.3 inhibits C/EBP alpha-dependent transcription that correlates with a block in granulocytic differentiation in vitro (Westendorf et al., 1998). Inhibition of C/EBP alpha function in these experiments was related to the direct association of AML1-ETO with C/EBP alpha. Mice that develop in the absence of C/EBP alpha lack neutrophils and are blocked in granulocytic development at the myeloblast stage (Zhang et al., 1997). Significant down-regulation of C/EBP alpha has also been seen in patient samples bearing the t(8;21) translocation, thus establishing C/EBP alpha as a potentially critical target gene in AML1-ETO-associated leukemia (Pabst et al., 2001a; Pabst et al., 2001b).

C/EBP alpha is a transcription factor with an important role in granulocyte development (for review, see Tenen et al., 1997). C/EBP alpha can interact with a number of transcription factors that control HSC differentiation, including NF-kB and Re1 proteins, members of the CREB/ATF family, Sp1, RB, and members of the fos/jun zipper family. PU.1 can physically interact with C/EBP alpha. Another functionally important interaction relevant to myeloid gene regulation involves C/EBP alpha and AML1, which regulates the promoter of M-CSF receptor gene (Zhang et al., 1996). C/EBP alpha is specifically expressed in human myelomonocytic cell lines and not in human erythroid, B-cell, or T-cell lines. In studies of murine 32D cells and human leukemic lines, such as HL-60 and U937 cells, C/EBP alpha was highly expressed in proliferating myelomonocytic cells upon induction of differentiation, and was down regulated with maturation. Northern blot analysis of mature peripheral blood neutrophils shows high levels of C/EBP alpha mRNA, which was undetectable in adherent peripheral blood monocytes, suggesting that C/EBP alpha might be important in neutrophilic but not monocytic lineage development. C/EBP alpha has been shown to regulate granulocytic differentiation at least through the up-regulation of the G-CSFR, IL-6R, and MPO (Zhang et al., 1998). Although a clear expression analysis of C/EBP alpha has not been done on HSCs, nor has an analysis been done of the HSC compartment in C/EBP alpha knockout animals, the above-mentioned studies indicate that C/EBP alpha regulates and promotes differentiation of a number of cell types (from primitive myeloblasts to more differentiated neutrophils) and it is also inhibited by the activity of AML1-ETO. It is therefore a possible target gene in HSC that might play a role in HSC self-renewal.

PU.1 is a transcription factor that has also been implicated in the differentiation of both myeloid and lymphoid lineage cells (reviewed in Fisher and Scott, 1998). It is necessary for the normal formation of both lymphoid and myeloid cells in vivo based on PU.1 gene knockout experiments (Scott et al., 1994). Studies from Applicant's lab have shown that in the absence of PU.1, there are no detectable HSC within the fetal liver of developing mouse embryos, with may suggest that PU.1 is responsible for the maintenance or self-renewal of HSC (H. Kim and C. Klug, submitted manuscript). AML1 can directly bind PU.1 (as can C/EBP alpha and the AML1-ETO translocation protein). It is also expressed in cells that have the Sca-1⁺c-kit⁺Lin⁻ Thy-1,1^{1o} phenotype based on observations from the Applicant's laboratory.

The absence of either AML1, C/EBP alpha, and/or PU.1 activity inhibits the ability of precursor cells to differentiate even in the presence of the proper differentiation signals within animals that lack these factors. By restoring activity of these factors, precursor cells can then be stimulated to undergo normal differentiation in response to the appropriate signals.

The present disclosure shows that while modulating of the function of the target factors reduces the differentiation potential of HSC, the ability of HSC to divide (self-renewal) is not adversely effected. In one embodiment, it is shown that inhibition of the target factors AML1, C/EBP alpha and/or PU.1 reduces the differentiation potential of HSC, the ability of HSC to divide (self-renewal) is not adversely effected. Therefore, antagonists of target factors, such as, but not limited to, AML1, C/EBP alpha, PU.1, may be used to modulate the activity of cellular mechanisms that regulate HSC differentiation and/or self-renewal in a manner that mimics the function of AML1-ETO. However, modulation of target factors should not be limited to inhibition of the function of the target factors. Modulation may occur as a result of increasing the function of the target factors or by translocating the function of the target factors to a different area of the cell.

Modulation of the function of target factors, such as AML1, C/EBP alpha, and/or PU.1, may be achieved in many different ways. The following examples are provided as specific to AML1, C/EBP alpha, and/or PU.1 and provide that the modulation of function is an inhibition of fuction. Inhibition may occur at the level of synthesis of these factors, interaction with cellular factors required for basal activity or enhanced activity, such as cofactors (AML1/C/EBP alpha/PU.1 interactions), interactions with their DNA binding motifs (transcription factor-DNA interactions), or by targeted degradation or inhibition of their mRNAs using methods like small interfering RNAs (RNAi) (Elbashir et al., 2001) or peptide nucleic acids (Ray and Norden, 2000). These inhibitions may be direct or indirect. For example, direct inhibition of AML1 interactions may occur through the use of a pharmacologic agent to interact with AML1, thereby blocking the association of AML1 with cellular factors. Indirect inhibition of AML1 interactions may be the use of a pharmacologic agent to block the production of the cellular factor, thereby obviating the ability of AML1 to bind to the cellular factor. These approaches may be used alone or in any combination. Specific examples of methods include blocking the transcription or translation of the AML1 protein, using oligonucleotides that mimic the binding sites of the AML1 protein to sequester AML1 in non-functional complexes (meaning that the sequestered AML1 is not available for stimulation of transcription), pharmacological inhibition of AML1 activity, inhibiting the binding or production of accessory proteins required for AML1 activity, or stimulating the activity of related members of the AML1 family such that factors required for AML1 activity are not present in sufficient levels for AML1 function. Other methods for inhibiting AML1 activity may also be used, with the above methods provided by way of example only. The methods and reasoning above, although described in reference to AML1, may be used to inhibit other targets of AML1-ETO including, but not limited to, C/EBP alpha, and/or PU.1.

The inhibition of AML1, C/EBP alpha, and/or PU.1 activity may be of any desired period and may be done using pharmacologic agents or through the use of recombinant vectors to transiently inhibit the activity of these proteins during in vitro expansion protocols. AML1 C/EBP alpha, and/or PU.1 function may be restored by removal of the antagonist.

Isolation of HSC

In order to practice the method of the present disclosure, HSC populations must be obtained and treated so as to inhibit the activity of AML1, C/EBP alpha, PU.1, or other target factors identified as misregulated by AML1-ETO. HSC may be isolated from a number of primary tissue sources including mouse, mammalian or human bone marrow, human cord blood, or mobilized peripheral blood CD34⁺ or CD34⁻ progenitor cell populations. Stem cells associated with other tissues including, but not limited to, pancreas, muscle, nervous tissue, skin, and adipose tissue may also be used. In addition, HSC may be purified to some degree (like human CD34⁺CD38⁻ cells), or unpurified populations of cells containing HSC may be used. In order to inhibit the activity of a specific factor(s) in HSC, it is not necessary that the precursor cell populations are a pure population, although some degree of purification may be useful to keep cell culture volumes to a minimum during in vitro expansion. HSC will be transiently treated with inhibitors of the target factors until the desired degree of expansion is achieved. More permanent genetic modifications of HSC, like the use of an AML1-ETO-ER retrovirus (described in Examples 1 and 11) that stably integrates into the target cell genome may be used. These approaches may require excision of the integrated, exogenous DNA via standard recombinase approaches like the activation of Cre recombinase to delete a DNA fragment that was flanked by loxP sites. This is necessary to eliminate any toxicity or oncogenicity associated with in vitro treatment approaches that are not transient by nature.

The isolation of precursor cells for use in the present disclosure can be carried out by any of numerous methods commonly known to those skilled in the art. For example, one common method for isolating precursor cells is to collect a population of cells from a subject and using fluorescence activated cell sorting (FACS) to separate the desired cells based on the differential expression of specific antigens that have been bound by fluorescent-tagged antibodies. Techniques include (a) the isolation and establishment of HSC cultures from bone marrow cells isolated from the future host subject (autologous cells), or a donor that is not the host subject, or (b) the use of NOD-SCID mice to expand HSC in an animal model for human hematopoiesis, which may be syngeneic, allogeneic or xenogeneic. When allogeneic or xenogenic HSC are used, it is common to use a method of suppressing transplantation immune reactions of the future host subject in conjunction with the administration of the xenogenic cells. In one embodiment, this approach will involve the expansion of autologous cells obtained from the individual who will ultimately be the recipient of expanded stem cell product, unless there are genetic abnormalities of such HSC that are specific to the donor that would preclude their use in either expansion protocols or for treatment applications.

The expansion of HSC and/or their progeny can be assessed by techniques well known in the art, such as in vivo reconstitution of NOD-SCID mice for human HSC expansion. Additional in vitro surrogate assays would include spleen colony-forming assays, cobblestone area-forming cell assays, and long-term culture initiating cell assays.

Expansion and Differentiation of Precursor Cells

After the precursor cells have been isolated according to the methods described above, or other methods known in the art, HSC may be exposed to an inhibitor of AML1, C/EBP alpha, PU.1, or other target factors misregulated by AML1-ETO so as to allow increased self-renewal and decreased differentiation as described above. These cells are exposed to appropriate cell growth conditions such that the precursor cells can undergo self-renewal in the presence of the inhibitors without differentiation caused by exogenous cytokine conditions used in the media to inhibit apoptosis of HSC. In this manner, an expanded cell population can be obtained. We have defined such in vitro cytokine conditions that allow for minimal differentiation and modest HSC expansion (2-3-fold) over an in vitro culture period of three weeks, which was the maximal time tested. These conditions include the use of stem cell factor, interleukin 6, leukemia inhibitory factor, bone morphogenic protein 2, serum-free culture media, and a supportive extracellular matrix substrate like fibronectin. The extent of HSC expansion is monitored by in vivo transplantation of cultured cells. Once the HSCs have been expanded to a desired level, the inhibitor of AML1, C/EBP alpha, PU.1, or other target factor can be removed. Removing the inhibitor restores wild-type cellular activity to the expanded cells to allow for in vivo differentiation.

Inhibition of Target Factor Activity

In one embodiment, the means for inhibiting activity of AML1, C/EBP alpha, PU.1, or other target protein will be through the use of RNA interference (RNAi). In this approach, small double-stranded complementary oligonucleotides will be used to target transient degradation of specific mRNA species in HSC. A panel of oligonucleotides complementary to different portions of the target mRNA species will be utilized to establish the sequences that induce a maximal degradation response. Multiple RNAi species can be used simultaneously to target degradation of AML1, C/EBP alpha and/or PU.1 either alone or in various combinations. Since the oligonucleotides have a limited half-life, they will induce a transient degradation response. This will provide a means to conditionally inactive any protein for a short duration of time during in vitro expansion. The RNAi sequences may be introduced into HSC via non-replicating viral vectors that remain episomal within target cells and express the RNAi sequences. An example of such a vector would include an adenoviral delivery system, where small hairpinned mRNA species could be expressed from an internal RNA polymerase III promoter that does not stimulate polyadenylation of transcribed RNA species. As mentioned previously, other means of selectively inhibiting the activity of AML1, C/EBP alpha, PU.1, or other target protein in a transient manner will include the use of double-stranded DNA sequences that represent the DNA-binding sites for AML1, C/EBP alpha and/or PU.1. This “decoy” approach acts to sequester transcription factors away from their target sequences within genomic DNA by providing a vast excess of DNA-binding sequence. These and other approaches mentioned above will be pursued in parallel to determine optimal HSC expansion conditions that minimize differentiation and cellular toxicity while allowing proliferation and self-renewal.

Pharmaceutical Compositions

The disclosure also provides methods of treatment by administration to a subject of a pharmaceutical composition comprising a therapeutically effective amount of HSCs and/or precursor cells that have been treated (as described above) to modulate the activity of proteins involved in the regulation of self-renewal or differentiation to induce expansion (therapeutic precursor cells). These therapeutic precursor cells may be purified to some degree or used in a mixed population of cells without purification. In one embodiment, the therapeutic precursor cells administered to the subject are HSCs. In an alternate embodiment, the therapeutic precursor cells administered to the subject are hematopoietic progenitor cells, or a combination of hematopoietic progenitor cells and HSCs. In addition, the therapeutic precursor cells may be modified to express recombinant gene products, as would be the case if cells were used for gene therapy applications. The subject is preferably an animal, including but not limited to animals such as cows, pigs, horses, chickens, cats, dogs, etc. In one embodiment, the subject is a mammal. In an alternate embodiment, the subject is a human.

The pharmaceutical compositions of the present disclosure comprise a therapeutically effective amount of therapeutic precursor cells, and a pharmaceutically acceptable carrier or excipient. Such a carrier includes but is not limited to saline, buffered saline, dextrose, water, glycerol, ethanol, and combinations thereof. The pharmaceutical composition may be sterile. The formulation of the pharmaceutical composition should suit the mode of administration. The pharmaceutical composition, if desired, can also contain minor amounts of wetting or emulsifying agents, or pH buffering agents. The pharmaceutical composition can be a liquid solution, suspension, or emulsion.

In one embodiment, the pharmaceutical composition is formulated in accordance with routine procedures as a pharmaceutical composition adapted for intravenous administration to humans. Typically, compositions for intravenous administration are solutions in sterile isotonic aqueous buffer. Where necessary, the composition may also include a solubilizing agent and a local anesthetic such as lignocaine to ease pain at the site of the injection. Examples of alternate carriers and methods of formulation may be found in Remington The Science and Practice of Pharmacy (20^{th} edition). The pharmaceutical compositions of the present disclosure are administered to a subject in a therapeutically effective amount. The effective amount may vary according to a variety of factors such as the individual's condition, weight, sex and age. Other factors include the mode or site of administration. The pharmaceutical compositions may be provided to the individual by a variety of routes such as subcutaneous, topical, oral, intraosseous, intravenous, and intramuscular. Therapeutic precursor cells identified according to the methods disclosed herein may be used alone at appropriate dosages defined by routine testing in order to obtain optimal activity, while minimizing any potential toxicity. In addition, co-administration or sequential administration of other agents may be desirable. Therapeutic doses of therapeutic precursor cells would be determined primarily by the application.

Administration to Subject

In one embodiment, the subjects to which the cells are administered are immunocompromised or immunosuppressed or have an immune deficiency. For example, the subject may have Acquired Immune Deficiency Syndrome or have been exposed to radiation or chemotherapy regimens for the treatment of cancer, and the subjects are administered therapeutic precursor cells such that the administered cells perform a needed immune or hematopoietic function. Numerous other examples for uses of expanded cells would include all applications where “transdifferentiation” of HSC would be beneficial. That is, in tissue replacement therapies where HSC differentiate into hepatocytes or neural tissue that has been damaged by disease or injury. Additionally, applications primarily targeted to bone marrow transplantation and gene therapy would be used for hematopoietic replacement.

Synopsis

The present disclosure has described the phenotype of HSCs that express the AML1-ETO chimeric protein that is found in association with a particular form of acute myeloid leukemia in man. It was found that AML1-ETO caused HSC to significantly expand (as much as 100-fold) in vivo and that this expansion can also be accomplished in vitro. Expansion of HSC was accompanied by a reduced tendency for HSC to differentiate without inhibition of cellular proliferation (i.e. self-renewal), which indicates that AML1-ETO is regulating/modulating the function of factors involved in differentiation of HSC and/or promoting factors that stimulate self-renewal. The extent of HSC expansion in the presence of AML1-ETO can have significant therapeutic applications, especially since many stem cell sources are limited in therapeutic utility (like cord blood HSC) because of low HSC numbers within these tissues. Furthermore, expanded HSC can potentially open new doors to therapies requiring transdifferentiation of HSC into other tissues, which has been an inefficient process that is severely (or entirely) limited by HSC numbers obtained from any given donor. The target factors for AML1-ETO include, but are not limited to, the transcription factors AML1, C/EBP alpha, and/or PU.1. These proteins are all directly bound and inhibited by AML1-ETO in hematopoietic cells and each is known to induce hematopoietic differentiation and is expressed in multipotential progenitor cells. Although AML1, C/EBP alpha and PU.1 represent likely targets of AML1-ETO, this disclosure focuses on the entire set of self-renewal factors that are being regulated by AML1-ETO in stem cells. By targeting both known and yet to be identified factors in the self-renewal pathway being affected by AML1-ETO, therapeutic expansion of HSC may now be an achievable goal.

### EXAMPLES

### Example 1

### Generation of a AML1-ETO Expressing Hematopoietic Stem Cells

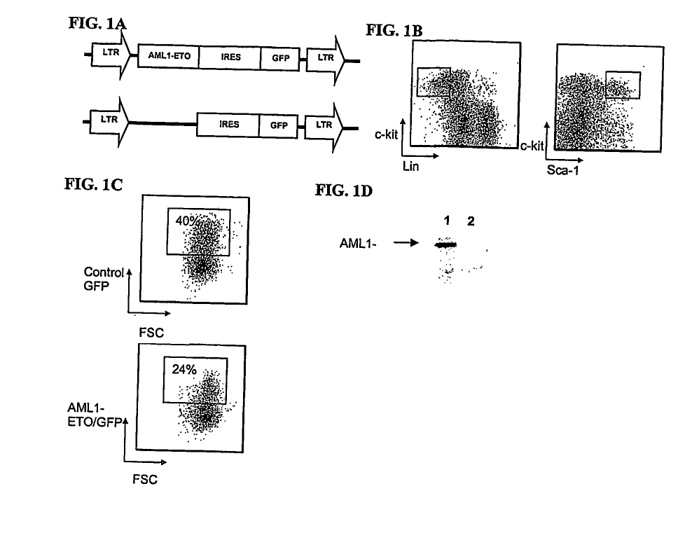

HSC of the phenotype c-kit+Sca-1+Lin− were double-sorted to a purity of >98% (FIG. 1B) and then transduced with retroviral supernatant containing either the control or AML1-ETO vectors (illustrated in FIG. 1A). Each vector was derived from the murine stem cell virus (MSCV) and contained an internal ribosome entry site (IRES) to allow for co-expression of the green fluorescent protein (GFP). Transduction efficiencies for the AML1-ETO virus ranged from 20-28% for the AML1-ETO/GFP virus and 30-40% for the control virus (FIG. 1C). Transduced HSC isolated from C57B6-Ly-5.2 mice were then re-sorted for GFP expression and then transplanted into lethally irradiated, congenic C57B6-Ly-5.1 animals at a dose of approximately 300 GFP+ cells per recipient.

AML1-ETO-expressing animals were also generated by transplanting retrovirally transduced whole bone marrow cells isolated from 5-fluorouracil-treated animals.

Expression of AML1-ETO from the retroviral vector was confirmed by Western blot analysis using a polyclonal anti-AML1 antibody and GFP+ myeloid-lineage cells sorted from the bone marrow of an 8-week post-reconstituted AML1-ETO animal (FIG. 1D, lane 1). GFP-negative cells contained no AML1-ETO protein (FIG. 1D, lane 2). The anti-AML1antibody was raised against a peptide encoding residues 32-50 of the human AML1 protein (10). The immunizing peptide has three amino acid differences between the murine and human sequence so a direct comparison between the levels of retrovirally-expressed AML1-ETO and endogenous AML1 protein in myeloid-lineage cells is not possible.

Other means to generate AML1-ETO expressing cells may also be used. Importantly, regulated expression of AML1-ETO may also be obtained in vitro using an AML1-ETO-ER fusion protein. In this case, AML1-ETO was fused in frame to the ligand binding domain of the estrogen receptor (Heyworth et al., 1999). This construct allows conditional regulation of the AML1-ETO protein such that in the presence of 4-hydroxytamoxifen (4-HT), AML1-ETO will be active due to its ability to translocate to the nucleus (leading to HSC expansion in vitro) and in the absence of 4-HT, the AML1-ETO protein will be sequestered to the cytoplasm in an inactive state. Expression of AML1-ETO-ER in HSC in vitro in the presence of 4-HT maintains and expands the ability of HSC to long-term reconstitute lethally-irradiated animals in an in vivo transplantation assay, which is the only true measure of HSC activity.

Therefore, inhibition of the downstream target factors of AML1-ETO (including, but not limited to, AML1, C/EBP alpha, and/or PU.1) should also promote the same in vitro self-renewal outcome and generate cells that can be used in a therapeutic context.

### Example 2

### Abnormal Myelopoiesis in AML1-ETO/GFP+ Peripheral Blood Cells

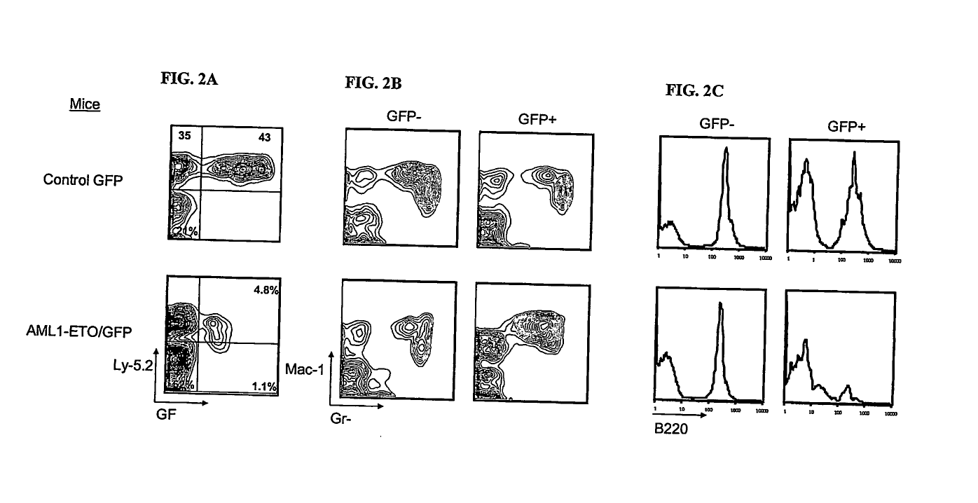

The effect of the AML1-ETO fusion protein on hematopoiesis was monitored in AML1-ETO-expressing and control GFP-expressing animals by flow cytometric analysis (FACS) of peripheral blood. All AML1-ETO (n=29) and control GFP (n=26) recipients were reconstituted with up to 85% of peripheral blood cells expressing the Ly-5.2 donor marker (FIG. 2A). Donor cells that silenced expression of the GFP marker were present in all reconstituted animals. Peripheral blood myeloid cells were analyzed by co-staining with Mac-1 (CD11b) and Gr-1. AML1-ETO/GFP+ cells showed an abnormal Mac-1/Gr-1 phenotype in all AML1-ETO mice compared to control GFP mice or to non-AML1-ETO-expressing cells (GFP−) within the AML1-ETO mice (FIG. 2B). Notably absent in the AML1-ETO/GFP+ population was a subset of Mac-1^{1o}Gr-1^{h1} cells that represents an essentially pure population of mature neutrophils. In addition, there was an over-representation of a unique subset of cells that expressed high levels of Mac-1 and intermediate levels of Gr-1. This subset of cells was present in the peripheral blood of all AML1-ETO mice at all time points analyzed and did not increase in frequency between 2-10 months post-reconstitution (n=3 for animals analyzed out to 10 months and n=26 for animals analyzed between 1 and 6 months post-transplant). In all analyses, the GFP− cells within the AML1-ETO mice resembled the GFP− and GFP+ cell profiles from control animals.

### Example 3

### Decreased B Lymphopoiesis in AML1-ETO/GFP+ Peripheral Blood Cells

Peripheral lymphoid cells in transplant recipients were analyzed by staining for B220 and CD3 expression on B and T cells, respectively. Analysis of the B220+ population in AML1-ETO and control GFP mice showed that B220 expression was significantly lower in AML1-ETO/GFP+ cells compared to controls (FIG. 2C). The number of cells expressing CD3 was dramatically decreased in AML1-ETO/GFP+ cells, although this observation was also seen in some of the control GFP+ animals, thus making it difficult to draw definitive conclusions on the role of AML1-ETO in T cell development at this point.

### Example 4

### Abnormal Myelopoiesis in AML1-ETO-Expressing Bone Marrow Cells

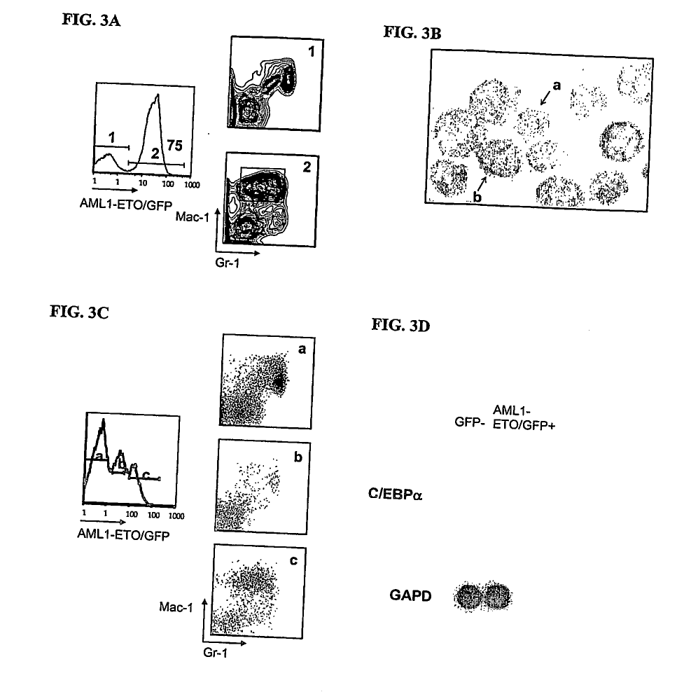

Given the abnormal myeloid phenotype in AML1-ETO/GFP+ peripheral blood cells, AML1-ETO-expressing mice were sacrificed to further investigate myeloid development in the bone marrow. AML1-ETO mice were sacrificed at 10-months post-transplant and analyzed for myeloid abnormalities by Mac-1/Gr-1 staining. All AML1-ETO mice (n=3) exhibited the same Mac-1^{hi}Gr-1^{int} population in the majority of AML1-ETO/GFP+ bone marrow cells compared to GFP− control myeloid cells analyzed from the same bone marrow (FIG. 3A). The appearance of this abnormal population in bone marrow was dependent on the level of AML1-ETO expression, as demonstrated by an AML1-ETO mouse that expressed both low and high levels of GFP (FIG. 3C). The dose-dependent phenotype in the myeloid lineage was not unexpected since AML1-ETO functions as a dominant inhibitor of normal AML1 activity.

In order to determine the morphology and function of the cells residing in the Mac-1^{hi} Gr-1^{int} population, we sorted these cells for Wright-Giemsa staining and for assays of myeloid colony-forming potential in methylcellulose. We observed no myeloid colony-forming activity in the Mac-1^{hi}Gr-1^{int} population when 2,000 of these cells were plated in triplicate in methylcellulose over a 10-day in vitro culture period. Plating 1,000 control myeloid-lineage cells isolated from the bone marrow of C57B6 animals gave rise to 1-10 myeloid colonies (see below). Wright-Giemsa cytospin preparations indicated that 95% of the Mac-1^{hi}Gr-1^{int} cells were metamyelocytes and immature band-form neutrophils (FIG. 3B), which is consistent with the lack of myeloid-colony forming activity in the population. In addition, there were no observed myeloblasts or promyelocytes in counts of 1,000 Mac-1^{hi}Gr-1^{int} cells from 10 independent microscope fields from two animals. In the animal shown in FIG. 3A, 38% of the total marrow was comprised of this myeloid subset. The other animals analyzed had 8% and 14% of Mac-1^{hi}Gr-1^{int} cells in the bone marrow at 10-months post-transplant. Interestingly, morphologic characterization of bone marrow from human patients with the t(8;21) translocation also showed abnormal nuclear condensation at the metamyelocyte stage.

### Example 5

### C/EBP Alpha Expression is Decreased in AML1-ETO-Expressing Bone Marrow Cells

Recent studies have demonstrated that AML1-ETO down-regulates transcription of C/EBP alpha, a transcription factor necessary for granulocytic differentiation, in patients with t(8;21)-associated leukemia. To determine whether C/EBP alpha expression was affected in AML1-ETO/GFP+ cells, RNA was isolated from FACS-sorted, myeloid AML1-ETO/GFP+ and myeloid GFP− cells from the same AML1-ETO-expressing animal. Northern analysis showed that the level of C/EBP alpha mRNA expression in AML1-ETO-expressing cells was 2.5-fold lower than in GFP− myeloid-lineage cells (FIG. 3D). These results confirm that AML1-ETO expression causes a down-regulation of C/EBP alpha levels in myeloid-lineage cells.

### Example 6

### Increased Myeloid Progenitors in the Presence of AML1-ETO

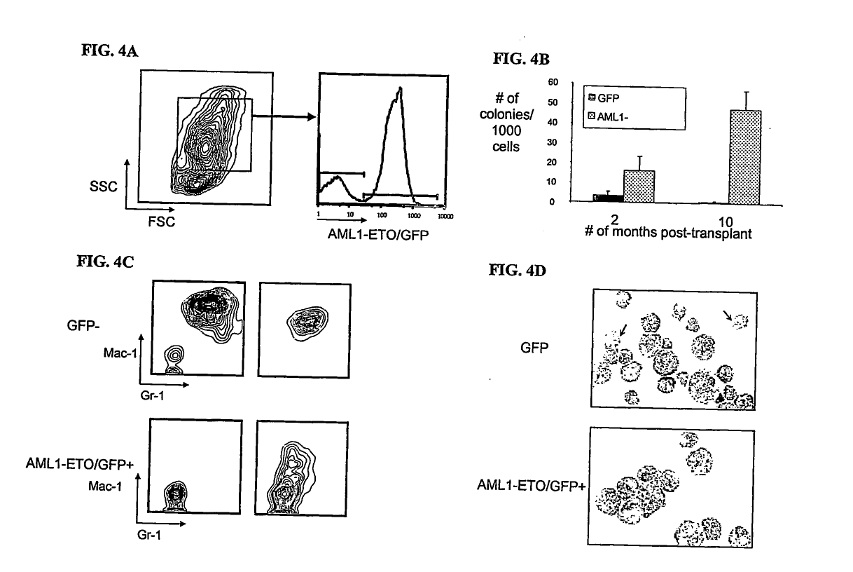

Changes in the number of myeloid progenitors in bone marrow were determined by in vitro colony-forming cell assays using GFP+ and GFP− cells isolated from AML1-ETO mice at 2 and 10 months post-transplant. One thousand myeloid scatter-gated AML1-ETO/GFP+ or GFP− cells from the same animal were sorted and then cultured in methylcellulose for 10 days (FIG. 4A). AML1-ETO/GFP+ cells isolated from 2-month post-transplant animals (n=3) gave rise to an average of 16 myeloid colonies per 1,000 cells plated in triplicate compared to GFP− cells, which averaged 4 myeloid colonies per 1,000 cells plated (FIG. 4B). The 4-fold increase in progenitor numbers compared to controls was statistically significant (p<0.001) and most likely represents a conservative estimate of progenitor cell expansion in the AML1-ETO/GFP+ fraction of marrow. This is based on the observation that the major myeloid subset (Mac-1^{hi}Gr-1^{int}) has no colony-forming activity and equivalent numbers of AML1-ETO/GFP+ and control GFP− myeloid cells were used in the plating.

The expansion of myeloid progenitors was further increased in the bone marrow of 10-month post-transplant animals (n=3), where 1,000 AML1-ETO/GFP+ cells gave rise to an average of 48 myeloid colonies compared to an average of 1 myeloid colony in GFP− control cells (FIG. 4B). Again, this 50-fold increase in the number of myeloid progenitors in AML1-ETO/GFP+ cells most likely represents a conservative estimate of the overall expansion. The percentages of total myeloid cells in bone marrow (GFP+ and GFP−) were 58, 41 and 72% from the 3 AML1-ETO 10-month animals. The percentages of GFP+ myeloid cells in the same animals were 44, 46, and 91%, respectively. This indicates that there was not preferential expansion of GFP+ myeloid-lineage cells in these animals (except in the latter case) even though the frequencies of specific myeloid subpopulations were significantly altered in cells that expressed AML1-ETO.

### Example 7

### The Presence of AML1-ETO Alters Myeloid Cell Differentiation

Wright-Giemsa stained cytospins of colonies derived from AML1-ETO/GFP+ cell platings showed a mixed lineage phenotype that included immature myeloid cells and mature macrophage (FIG. 4D). There were no segmented neutrophils present in AML1-ETO-expressing colonies. In contrast, cytospin preparations of GFP− colonies showed a number of mature segmented neutrophils (see arrows on FIG. 4D). FACS analysis of individual colonies stained with Mac-1 and Gr-1 confirmed that GFP− colonies were almost completely differentiated (9/10 colonies were Mac-1⁺Gr-1⁺). In contrast, AML1-ETO/GFP+ colonies remained primarily undifferentiated, with negative or low-level expression of Mac-1 in only a fraction of the cells from a single colony (FIG. 4C).

To assess the percentages of myeloid cell types in the bone marrow of the 3 animals used for methylcellulose assays at 10 months post-transplant, myeloid-gated GFP+ and GFP− cells were cytospun and stained with Wright-Giemsa. The 3 AML1-ETO/GFP+ fractions of marrow were highly shifted in representation toward primitive myeloid cell types, with 17, 48, and 21% myeloblast/promyelocytes compared to 1, 3, and 3% of the same cell subsets in the GFP− controls, respectively (Table 1). Overall, the frequency of myeloblast/promyelocytes in bone marrow of the 3 AML1-ETO animals (after normalization for the total percentage of GFP+ myeloid cells) was 4.6, 9.5, and 14.0%. These results support the data from the in vitro colony-forming cell assays, indicating that a substantial increase in myeloid progenitor populations have occurred by 10 months post-transplant in the AML1-ETO animals. One criteria used in the characterization of AML in humans is the presence of greater than 20% myeloblasts in bone marrow (11). Although the percentage of myeloblasts/promyelocytes in the 10-month post-transplant, AML1-ETO animals was not 20%, the results clearly indicate that a highly abnormal condition exists in the myeloid lineage that becomes more pronounced over time. The lack of leukemia in the AML1-ETO animals was further supported by bone sections characterized at 4 months post-transplant, which did not show evidence of granulocytic foci. This was also true of the spleen and liver at this stage.

### Example 8

### Expansion of HSC in AML1-ETO-Expressing Mice

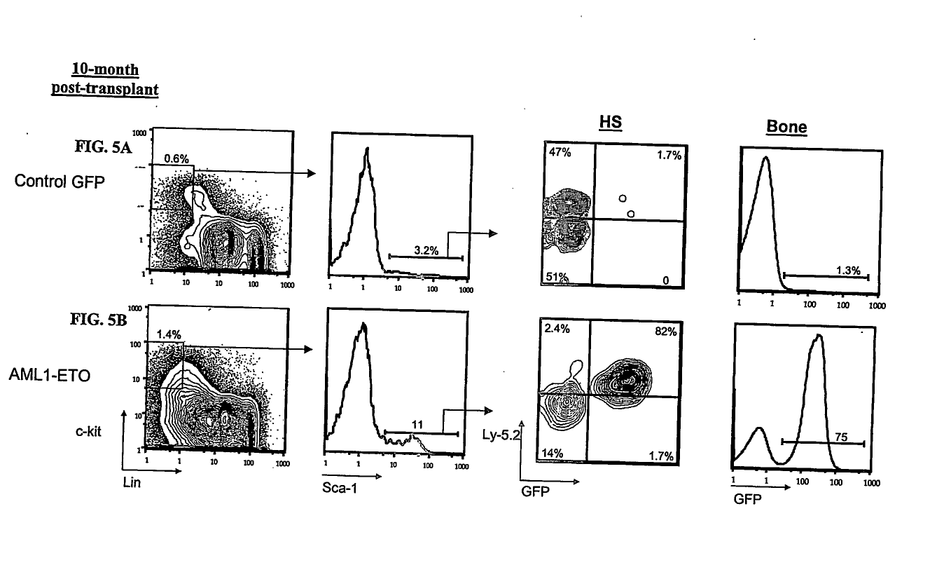

In order to characterize the HSC compartment in reconstituted animals, a 5-color FACS analysis of bone marrow isolated from animals transplanted with cells expressing either the AML1-ETO or control GFP vector (FIG. 5) was performed. HSC in reconstituted animals have the same cell-surface phenotype (c-kit+Sca-1+Lin−) as HSC isolated from un-manipulated bone marrow. Bone marrow cells isolated from the tibias and femurs were quantitatively harvested and counted prior to staining to determine absolute HSC numbers. FACS analysis was performed at 2 and 10 months post-transplant of purified/transduced HSC and at 2.5 months post-transplant of transduced whole bone marrow cells isolated from 5-fluorouracil-treated animals (Table 2). The latter samples were analyzed to determine whether HSC expansion and absolute number would be influenced by the presence of approximately 1×10⁶ bone marrow cells that were co-transduced and injected with HSC.

FIGS. 5A and 5B show a representative analysis and gating of one control GFP and one AML1-ETO animal analyzed at 10 months post-transplant, respectively. Table 2 summarizes the results from 8 AML1-ETO and 8 control animals analyzed at the indicated time points. There was a modest expansion (3-fold) in the absolute number of HSC in AML1-ETO-expressing animals at 2 months post-transplant and a dramatic expansion (29-fold) by 10 months. One animal at 10 months had more than 50 times the expected number of HSCs. HSC from AML1-ETO animals transplanted with co-cultured whole bone marrow cells were expanded 9.3-fold compared to control GFP animals at 2.5 months post-transplant. At every time point analyzed, the lowest number of HSC in an AML1-ETO animal was higher than the highest HSC number in any of the control GFP animals (Table 2).

The absolute number and frequency of HSC in control GFP animals was highly consistent in all animals, which suggests that the genetic control of hematopoietic stem cell pool size was maintained in primary transplant recipients expressing the control vector. In contrast, AML1-ETO-expressing HSC no longer seemed to be restricted by the regulatory mechanisms that influence homeostasis within the stem cell compartment. Consistent with this speculation was the observation that the increase in HSC number in the AML1-ETO animals was due to an expansion of AML1-ETO/GFP+ HSC within the HSC compartment. The percentages of AML1-ETO/GFP+ HSCs in the total HSC compartment ranged from 72-99% in 7/8 AML1-ETO animals (1 AM1-ETO animal had 44% GFP+ HSC), with a mean percentage of GFP+ HSC of 82% (n=8). This was in contrast to control GFP animals, where the mean percentage of GFP+ HSC was 15% (n=8). GFP− donor (Ly-5.2+) and recipient-type (Ly-5.2−) HSC were present in all animals.

### Example 9

### Delayed Differentiation in AML1-ETO-Expressing Hematopoietic Stem Cells

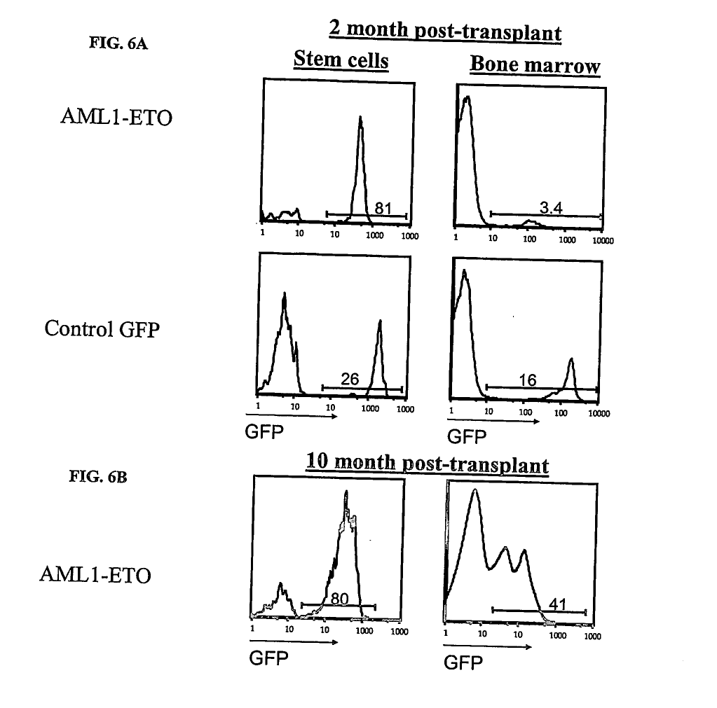

Despite the high percentage of AML1-ETO/GFP+ HSC at 2 months post transplant (75 and 81%, n=2), the percentage of AML1-ETO/GFP+ cells in the bone marrow was only 3.5 and 3.4%, respectively (FIG. 6A). In the control GFP animals, the percentage of GFP+ HSC more closely approximated the GFP percentage in the bone marrow. The delayed appearance of more differentiated GFP+ cells in bone marrow was consistent with a delay in the appearance of GFP+ peripheral blood cells in animals transplanted with AML1-ETO-transduced HSC (n=5 for AML1-ETO). In addition, AML1-ETO-expressing HSC were unable to radioprotect lethally irradiated recipient animals at a dose of 600 cells (n=6) whereas the same dose of control HSC radioprotected and reconstituted 4/5 animals. This supports the notion that AML1-ETO-expressing HSC have a reduced ability to differentiate and an enhanced tendency to undergo cell division events that favor self-renewal. In spite of an apparent partial block in differentiation at 2 months post-transplant, the percentage of GFP+ cells in older AML1-ETO-expressing animals increased to proportions seen in controls (FIG. 6B), which was largely due to an accumulation of GFP+ myeloid-lineage cells.

### Example 10

### Maintenance of Abnormal Myelopoiesis is Dependent on Sustained Expression of AML1-ETO in HSC

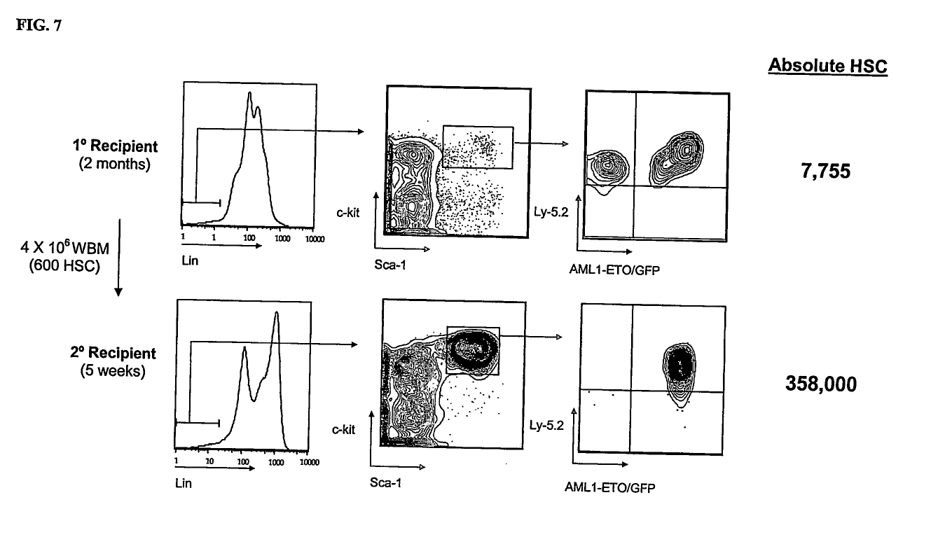

The lack of leukemia in AML1-ETO-expressing animals by 10 months post-reconstitution suggests that secondary mutations or additional time are necessary for disease progression. In an attempt to accelerate a disease phenotype, 4×10⁶ bone marrow cells from primary transplant recipients at either 2 or 10 months post-transplant were serially transplanted into multiple secondary recipient animals. Interestingly, only 1 out of 4 secondary recipients were reconstituted in bone marrow with AML1-ETO/GFP+ cells at 5 weeks post-transplant using marrow isolated from a 2-month primary donor, even though the bone marrow innoculum would have contained approximately 600 GFP+ HSC and about 114,000 GFP+ myeloid-lineage cells (FIG. 7 and Table 3). Of the 600 GFP+ HSC, 60 would be expected to re-home to the bone marrow and approximately 12 would re-home to the tibias and femurs, which represent about 20% of the total marrow cellularity. The 3 negative animals all showed high donor reconstitution and no GFP+ HSC, suggesting that donor GFP− HSC may have out-competed GFP+ HSC during engraftment or that GFP+ HSC homed less efficiently to marrow than GFP− HSC. The 1 animal that was donor reconstituted with AML1-ETO/GFP+ cells showed an enormous expansion of the HSC phenotype (from a predicted 12 HSC to 358,000 GFP+ HSC in both tibias and femurs in 5 weeks, FIG. 7). Approximately 33% of the total GFP+ cells in the marrow of this secondary recipient were c-kit+Sca-1+Lin−, supporting the observation that AML1-ETO-expressing HSC are partially blocked in their ability to differentiate. Of note was the lack of abnormal myelopoiesis in the absence of AML1-ETO/GFP+ HSC in the 3 negative secondary animals. This suggests that the 114,000 co-injected AML1-ETO/GFP+ myeloid-lineage cells do not extensively expand and retain a relatively short half-life in vivo.

Four secondary recipients derived from injection of 4×10⁶ bone marrow cells from a 10-month primary transplant animal were all highly reconstituted with AML1-ETO/GFP+ cells in peripheral blood for up to 6 months post-transplant (Table 3). One animal that was sacrificed at 2 months post-transplant had 21,134 total HSC, which represented a 33-fold expansion in HSC number over the 2-month reconstitution period. This was in contrast with the 30,000-fold expansion in 5 weeks seen in secondary recipient A3 (Table 3). The observation that 4/4 animals were highly reconstituted with AML1-ETO/GFP+ cells from a 10-month primary donor and only 1/4 secondary animals were reconstituted using the same number of bone marrow cells isolated from a 2-month donor may be related to the predicted number of GFP+ HSC in the inoculums. The GFP+ HSC number from the 10-month donor was approximately 32,000 cells, which was in contrast to the 600 GFP+ HSC from the 2-month primary donor. The total expansion of AML1-ETO/GFP+ HSC in vivo may be limited by some uncharacterized mechanism based on the observation that HSC expansion was more severely limited using bone marrow from primary animals that already displayed substantial HSC expansion (Table 3). This may indicate that the genetic mechanisms regulating the replicative lifespan of HSC are distinct from those that control the steady state number of stem cells in vivo.

### Example 11

### Expansion of HSC in vitro Using a Tamoxifen-Regulatable AML1-ETO-ER Fusion

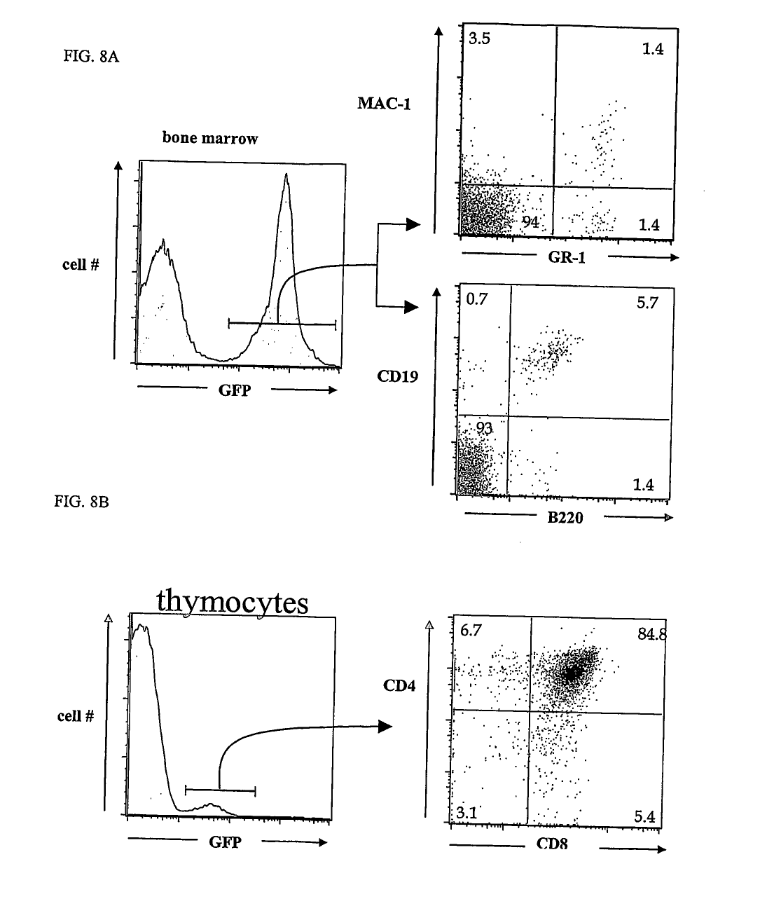

To test whether AML1-ETO could maintain or expand HSC numbers in vitro, AML1-ETO was fused to the hormone-binding domain of the estrogen receptor (ER). In the absence of inducer (4-HT), AML1-ETO will be sequestered in the cytoplasm, thus effectively inactivating AML1-ETO function. In the presence of 4-HT, AML1-ETO can translocate to the nucleus and act to repress transcription and stimulate self-renewal. Removal of 4-HT should then allow HSC to differentiate when the in vitro-expanded cells are used in the reconstitution of lethally irradiated mice. In this experiment, 400 HSC that were transduced with a retroviral vector that expressed AML1-ETO-ER were FACS-sorted into independent wells in the presence of serum-free media, the cytokines stem cell factor (at 50 ng/ml) and IL-6 (at 5 ng/ml), in the presence or absence of 4-HT. HSC that expressed the control GFP vector were similarly sorted as controls. In these culture conditions, it would be expected that all HSC activity would be lost within two weeks of culture as determined by their inability to long-term repopulate irradiated mice. Cells were cultured for 15 days with changes of media and replacement of cytokines every two days. After this time, decreasing fractions of the original well were transplanted into irradiated mice to test if HSC had self-renewed and not differentiated in the cultures. Analysis of the transplanted mice showed no peripheral blood reconstitution in any well where control HSC were expanded in the presence or absence of 4-HT (out of a total of 9 mice analyzed at doses that represented ½, 1/12, and 1/60^{th} of the original cells that initiated the experiment, Table 4). HSC that expressed AML1-ETO-ER in the absence of 4-HT were also lost in the culture time frame in all samples (a total of 8 mice). In contrast to these results, 2/3 mice were long-term reconstituted (greater than 4 months) with cells from wells where HSC expressed AML1-ETO-ER in the presence of 4-HT. Reconstitution of all lineages four months post-transplant; including B cells, T cells in the thymus, and myeloid-lineage cells is shown in FIGS. 8A and 8B, respectively. This indicates that the expanded HSC were truly pluripotential. These results show that AML1-ETO directly influences self-renewal of HSC, which was strongly suggested by the in vivo expansion data described above.

### METHODS

Generation of Retrovirus

AML1-ETO was cloned upstream of the IRES element into the EcoR1 site of the parental MSCV IRES GFP vector. Retroviral constructs were transiently transfected into BOSC23 ecotropic packaging cells by calcium phosphate co-precipitation. Viral supernatants were titered using NIH 3T3 cells. Titers ranged between 3×10⁶ and 1×10⁷ IU/mL.

HSC Isolation and Retroviral Transduction

An enriched population of HSC of the surface phenotype Sca-1⁺c-kit⁺Lin⁻ were isolated by FACS and pre-stimulated in cytokines as previously described. Bone marrow cells from 5-fluorouracil-treated mice (isolated 4 days post-IP injection of 150 mg/kg body weight of 5-FU) were treated with ACK (0.15M NH₄Cl and 0.01M KHCO₃) for 5 minutes on ice to lyse red blood cells and then pre-stimulated for 24 hours. After pre-stimulation, cells were co-cultured on transiently transfected and irradiated (30 Gy) BOSC23 cells in the presence of 4 μg/mL of polybrene for 48 hours prior to transplantation.

Transplantation

Congenic, C57B/6-Ly-5.1 mice (3-4 months of age) were used as transplant recipients. Prior to transplantation, Ly-5.1 mice were lethally irradiated with 10 Gy in a split dose separated by 3 hours. 300400 re-sorted GFP+/Ly-5.2+ cells and a radioprotective dose of 2×10³ Ly-5.1 bone marrow cells were transplanted into anesthetized mice by retro-orbital injection. 4×10⁶ bone marrow cells were used in serial transplant experiments and 1-6×10⁶ bone marrow cells were used in 5-FU transplants. Mice were maintained for 2-3 weeks on acidified water containing neomycin sulfate (1.1 g/L) and polymixin B sulfate (10⁶U/L) or sulfamethoxazole (400 mg/L).

Histology

For cytospin preparation, 4×10⁴ bone marrow cells in PBS/12% FCS or methycellulose colonies in 150 μl of Iscove's IMDM/12% FCS were centrifuged onto glass slides and stained with Wright-Giemsa. Blood and bone marrow counts were determined manually.

Myeloid Colony-Forming Assay

1,000 of each AML1-ETO/GFP+ or GFP− myeloid scatter-gated cells isolated from the same mouse were sorted into Iscove's IMDM media supplemented with 10% heat-inactivated FCS and then plated into MethoCult™ 3434 media (StemCell Technologies, Vancouver) supplemented with GM-CSF (0.5 ng/mL, R & D Systems). Colonies were typed at day 10.

Western Blot

Approximately 3×10⁶ myeloid scatter-gated cells were sorted as either AML1-ETO/GFP+ or GFP− from 2 AML1-ETO animals 3 months post-transplant. Cells were lysed in Laemmli buffer and run on a 10% polyacrylamide gel. AML-ETO was detected using a rabbit polyclonal antibody raised against a peptide encoding residues 32-50 of the human AML1 protein. The primary staining was visualized using a goat anti-rabbit HRP-conjugated secondary antibody and ECL (Amersham Pharmacia).

Northern Blot

Total RNA from approximately 8×10⁶ myeloid scatter-gated cells was isolated using RNA Stat-60 according to the manufacturers instructions (Tel-test “B”, INC. Friendswood, Tex.). Total RNA (7.5 μg) was run on a 1% agarose/0.6% formaldehyde gel, transferred to Hybond-N (Amersham) membrane, and hybridized according to the supplier's protocol. A murine GAPDH (Ambion) and C/EBP alpha probe (kindly provided by Dr. Dan Tenen, Harvard University) were used for detection.

All references to articles, books, patents, websites and other publications in this disclosure are considered incorporated by reference.

### REFERENCES

1. Bhardwaj, G., B. Murdoch, D. Wu, D. P. Baker, K. P. Williams, K. Chadwick, L. E. Ling, F. N. Karanu, and M. Bhatia. 2001. Sonic hedgehog induces the proliferation of primitive human hematopoietic cells via BMP regulation. Nat Immunol 2:172-180.

2. Bravo, J., Z. Li, N. A. Speck, and A. J. Warren. 2001. The leukemia-associated AML1 (Runx1)—CBF beta complex functions as a DNA-induced molecular clamp. Nat Struct Biol 8:371-378.

3. de Guzman, C. G., A. J. Warren, Z. Zhang, L. Gartland, P. Erickson, H. Drabkin, S. W. Hiebert, and C. A. Klug. 2002. Hematopoietic stem cell expansion and distinct myeloid developmental abnormalities in a murine model of the AML1-ETO translocation. Mol Cell Biol 22:5506-5517.

4. de Haan, G., and G. Van Zant. 1997. Intrinsic and extrinsic control of hemopoietic stem cell numbers: mapping of a stem cell gene. J Exp Med 186:529-536.

5. Dick, J. E., M. C. Magli, D. Huszar, R. A. Phillips, and A. Bernstein. 1985. Introduction of a selectable gene into primitive stem cells capable of long-term reconstitution of the hemopoietic system of W/Wv mice. Cell 42:71-79.

6. Downing, J. R. 1999. The AML1-ETO chimaeric transcription factor in acute myeloid leukaemia: biology and clinical significance. Br J Haematol 106:296-308.

7. Elbashir, S. M., J. Harborth, W. Lendeckel, A. Yalcin, K. Weber, and T. Tuschl. 2001. Duplexes of 21-nucleotide RNAs mediate RNA interference in cultured mammalian cells. Nature 411:494-498.

8. Erickson, P., J. Gao, K. S. Chang, T. Look, E. Whisenant, S. Raimondi, R. Lasher, J. Trujillo, J. Rowley, and H. Drabkin. 1992. Identification of breakpoints in t(8;21) acute myelogenous leukemia and isolation of a fusion transcript, AML1/ETO, with similarity to Drosophila segmentation gene, runt. Blood 80:1825-1831.

9. Evans, M. J., and M. H. Kaufman. 1981. Establishment in culture of pluripotential cells from mouse embryos. Nature 292:154-156.

10. Fisher, R. C., and E. W. Scott. 1998. Role of PU.1 in hematopoiesis. Stem Cells 16:25-37.

11. Hawley, R. G., F. H. Lieu, A. Z. Fong, and T. S. Hawley. 1994. Versatile retroviral vectors for potential use in gene therapy. Gene Ther 1:136-138.

12. Heyworth, C., K. Gale, M. Dexter, G. May, and T. Enver. 1999. A GATA-2/estrogen receptor chimera functions as a ligand-dependent negative regulator of self-renewal. Genes Dev. 13:1847-1860.

13. Lemischka, I. R., D. H. Raulet, and R. C. Mulligan. 1986. Developmental potential and dynamic behavior of hematopoietic stem cells. Cell 45:917-927.

14. Lutterbach, B., J. J. Westendorf, B. Linggi, A. Patten, M. Moniwa, J. R. Davie, K. D. Huynh, V. J. Bardwell, R. M. Lavinsky, M. G. Rosenfeld, C. Glass, E. Seto, and S. W. Hiebert. 1998. ETO, a target of t(8;21) in acute leukemia, interacts with the N—CoR and mSin3 corepressors. Mol Cell Biol 18:7176-7184.

15. Martin, G. R. 1981. Isolation of a pluripotent cell line from early mouse embryos cultured in medium conditioned by teratocarcinoma stem cells. Proc Natl Acad Sci USA78:7634-7638.

16. Meyers, S., J. R. Downing, and S. W. Hiebert. 1993. Identification of AML-1 and the (8;21) translocation protein (AML-1/ETO) as sequence-specific DNA-binding proteins: the runt homology domain is required for DNA binding and protein-protein interactions. Mol Cell Biol 13:6336-6345.

17. Miller, C. L., and C. J. Eaves. 1997. Expansion in vitro of adult murine hematopoietic stem cells with transplantable lympho-myeloid reconstituting ability. Proc Natl Acad Sci USA 94:13648-13653.

18. Miyamoto, T., I. L. Weissman, and K. Akashi. 2000. AML1/ETO-expressing nonleukemic stem cells in acute myelogenous leukemia with 8;21 chromosomal translocation. Proc Natl Acad Sci USA 97:7521-7526.

19. Miyoshi, H., K. Shimizu, T. Kozu, N. Maseki, Y. Kaneko, and M. Ohki. 1991. t(8;21) breakpoints on chromosome 21 in acute myeloid leukemia are clustered within a limited region of a single gene, AML1. Proc Natl Acad Sci USA 88:10431-10434.

21. Morrison, S. J. and I. L. Weissman. 1994. The long-term repopulating subset of hematopoietic stem cells is deterministic and isolatable by phenotype. Immunity 1:661-673.

22. Muller-Sieburg, C. E., and R. Riblet. 1996. Genetic control of the frequency of hematopoietic stem cells in mice: mapping of a candidate locus to chromosome 1. J Exp Med 183:1141-1150.

23. Mulloy, J. C., J. Cammenga, K. L. MacKenzie, F. J. Berguido, M. A. Moore, and S. D. Nimer. 2002. The AML1-ETO fusion protein promotes the expansion of human hematopoietic stem cells. Blood 99:15-23.

24.Nucifora, G., and J. D. Rowley. 1995. AML1 and the 8;21 and 3;21 translocations in acute and chronic myeloid leukemia. Blood 86:1-14.

25. Ogawa, E., M. Maruyama, H. Kagoshima, M. Inuzuka, J. Lu, M. Satake, K. Shigesada, and Y. Ito. 1993. PEBP2/PEA2 represents a family of transcription factors homologous to the products of the Drosophila runt gene and the human AML1gene. Proc Natl Acad Sci USA 90:6859-6863.

26. Okuda, T., Z. Cai, S. Yang, N. Lenny, C. J. Lyu, J. M. van Deursen, H. Harada, and J. R. Downing. 1998. Expression of a knocked-in AML1-ETO leukemia gene inhibits the establishment of normal definitive hematopoiesis and directly generates dysplastic hematopoietic progenitors. Blood 91:3134-3143.

27. Okuda, T., J. van Deursen, S. W. Hiebert, G. Grosveld, and J. R. Downing. 1996. AML1, the target of multiple chromosomal translocations in human leukemia, is essential for normal fetal liver hematopoiesis. Cell 84:321-330.

28. Pabst, T., B. U. Mueller, N. Harakawa, C. Schoch, T. Haferlach, G. Behre, W. Hiddemann, D. E. Zhang, and D. G. Tenen. 2001. AML1-ETO downregulates the granulocytic differentiation factor C/EBPalpha in t(8;21) myeloid leukemia. Nat Med 7:444-451.

29. Pabst, T., B. U. Mueller, P. Zhang, H. S. Radomska, S. Narravula, S. Schnittger, G. Behre, W. Hiddemann, and D. G. Tenen. 2001. Dominant-negative mutations of CEBPA, encoding CCAAT/enhancer binding protein-alpha (C/EBPalpha), in acute myeloid leukemia. Nat Genet 27:263-270.

30. Pear, W. S., G. P. Nolan, M. L. Scott, and D. Baltimore. 1993. Production of high-titer helper-free retroviruses by transient transfection. Proc Natl Acad Sci USA 90:8392-8396.

31. Ray, A. and B. Norden. 2000. Peptide nucleic acid (PNA): its medical and biotechnical applications and promise for the future. FASEB J.14:1041-1060.

32. Reynolds, B. A., and S. Weiss. 1992. Generation of neurons and astrocytes from isolated cells of the adult mammalian central nervous system. Science. 255:1707-1710.

33. Sasaki, K., H. Yagi, R. T. Bronson, K. Tominaga, T. Matsunashi, K. Deguchi, Y. Tani, T. Kishimoto, and T. Komori. 1996. Absence of fetal liver hematopoiesis in mice deficient in transcriptional coactivator core binding factor beta. Proc Natl Acad Sci USA 93:12359-12363.

34. Sauvageau, G., U. Thorsteinsdottir, C. J. Eaves, H. J. Lawrence, C. Largman, P. M. Lansdorp, and R. K. Humphries. 1995. Overexpression of HOXB4 in hematopoietic cells causes the selective expansion of more primitive populations in vitro and in vivo. Genes Dev 9:1753-1765.

35. Scott, E. W., M. C. Simon, J. Anastasi, and H. Singh. 1994. Requirement of transcription factor PU.1 in the development of multiple hematopoietic lineages. Science 265:1573-1577.

36. Tenen, D. G., R. Hromas, J. D. Licht, and D. E. Zhang. 1997. Transcription factors, normal myeloid development, and leukemia. Blood 90:489-519.

37. Thorsteinsdottir, U., A. Mamo, E. Kroon, L. Jerome, J. Bijl, H. J. Lawrence, R. K. Humphries, and G. Sauvageau. 2002. Overexpression of the myeloid leukemia-associated Hoxa9 gene in bone marrow cells induces stem cell expansion. Blood 99:121-129.

38. Varnum-Finney, B., L. Xu, C. Brashem-Stein, C. Nourigat, D. Flowers, S. Bakkour, W. S. Pear, and I. D. Bernstein. 2000. Pluripotent, cytokine-dependent, hematopoietic stem cells are immortalized by constitutive Notch1 signaling. Nat Med 6:1278-1281.

39. Wang, Q., T. Stacy, M. Binder, M. Marin-Padilla, A. H. Sharpe, and N. A. Speck. 1996. Disruption of the Cbfa2 gene causes necrosis and hemorrhaging in the central nervous system and blocks definitive hematopoiesis. Proc Natl Acad Sci USA 93:3444-3449.

40. Wang, Q., T. Stacy, J. D. Miller, A. F. Lewis, T. L. Gu, X. Huang, J. H. Bushweller, J. C. Bories, F. W. Alt, G. Ryan, P. P. Liu, A. Wynshaw-Boris, M. Binder, M. Marin-Padilla, A. H. Sharpe, and N. A. Speck. 1996. The CBFbeta subunit is essential for CBFalpha2 (AML1) function in vivo. Cell 87:697-708.

41. Wang, S., Q. Wang, B. E. Crute, I. N. Melnikova, S. R. Keller, and N. A. Speck. 1993. Cloning and characterization of subunits of the T-cell receptor and murine leukemia virus enhancer core-binding factor. Mol Cell Biol 13:3324-3339.

42. Weissman, I. L. 2000. Stem cells: units of development, units of regeneration, and units in evolution. Cell 100:157-168.

43. Westendorf, J. J., C. M. Yamamoto, N. Lenny, J. R. Downing, M. E. Selsted, and S. W. Hiebert. 1998. The t(8;21) fusion product, AML-1-ETO, associates with C/EBP-alpha, inhibits C/EBP-alpha-dependent transcription, and blocks granulocytic differentiation. Mol Cell Biol 18:322-333.

44. Yergeau, D. A., C. J. Hetherington, Q. Wang, P. Zhang, A. H. Sharpe, M. Binder, M. Marin-Padilla, D. G. Tenen, N. A. Speck, and D. E. Zhang. 1997. Embryonic lethality and impairment of haematopoiesis in mice heterozygous for an AML1-ETO fusion gene. Nat Genet 15:303-306.

45. Zhang, D. E., C. J. Hetherington, S. Meyers, K. L. Rhoades, C. J. Larson, H. M. Chen, S. W. Hiebert, and D. G. Tenen. 1996. CCAAT enhancer-binding protein (C/EBP) and AML1 (CBF alpha2) synergistically activate the macrophage colony-stimulating factor receptor promoter. Mol Cell Biol 16:1231-1240.

46. Zhang, D. E., P. Zhang, N. D. Wang, C. J. Hetherington, G. J. Darlington, and D. G. Tenen. 1997. Absence of granulocyte colony-stimulating factor signaling and neutrophil development in CCAAT enhancer binding protein alpha-deficient mice. Proc Natl Acad Sci USA 94:569-574.

47. Zhang, P., A. Iwama, M. W. Datta, G. J. Darlington, D. C. Link, and D. G. Tenen. 1998. Upregulation of interleukin 6 and granulocyte colony-stimulating factor receptors by transcription factor CCAAT enhancer binding protein alpha (C/EBP alpha) is critical for granulopoiesis. J Exp Med 188:1173-1184.

TABLE 1
Differential counts of sorted myeloid bone marrow cells from 10-month post-transplant
AML1-ETO animals

| GFP- | Cell types | | | | | |
|---|---|---|---|---|---|---|
| expressing | | | Meta + Band | | Mature Eosino | Eosino Myelo |
| cells | Blasts + Pro (%) | Mye (%) | (%) | Baso (%) | (%) | (%) |
| + | 17 | 12 | 69 | <1 | <1 | 2 |
| | 48 | 7 | 44 | <1 | <1 | 1 |
| | 21 | 6 | 73 | <1 | <1 | <1 |
| − | 1 | 7 | 89 | <1 | 3 | <1 |
| | 3 | 1 | 92 | <1 | 4 | <1 |
| | 3 | 8 | 89 | <1 | <1 | <1 |

The data reported here are percentages of > 300 cells counted per sample.
Blasts + Pro: myeloblasts and promyelocytes;
Mye: myelocytes;
Meta + Band: metamyelocytes and band nuclear granulocytes;
Baso: basophils;
Mature Eosino: mature eosinophils;
Eosino myelo: eosinophilic myelocytes.
Statistical analysis (t test) showed statistically significant differences between GFP− and AML1-ETO/GFP+ expressing cells in myeloblasts and promyelocytes (p < 0.05), metamyelocytes and band nuclear granulocytes (p < 0.04), and mature eosinophils (p < 0.01).

TABLE 2
Absolute number and frequency of hematopoietic stem cells in transplanted animals

| Time | AML1-ETO/GFP mice | | Control GFP mice | | |
|---|---|---|---|---|---|
| post-transplant | | Freq of HSC | Absolute HSC | Freq of HSC | Average fold |
| (mo) | Absolute HSC number | (%) | number | (%) | expansion |
| 2 | 7,755 | 0.017 | 1,509 | 0.004 | 3 |
| | 10,802 | 0.018 | 2,326 | 0.007 | |
| | | | 5,895 | 0.015 | |
| 10 | 93,960 | 0.116 | 6,162 | 0.011 | 29 |
| | 118,556 | 0.163 | 10,200 | 0.020 | |
| | 505,200 | 0.800 | | | |
| 2.5* | 52,100 | 0.146 | 10,980 | 0.015 | 9 |
| | 16,480 | 0.032 | 10,890 | 0.015 | |
| | 259,980 | 0.619 | 13,530 | 0.022 | |

Hematopoietic stem cells are derived from the femurs and tibias of transplanted mice. Average fold expansion is a multiple of the average HSC number in AML1-ETO transplanted animals over the average HSC number in control GFP transplanted animals at a given time point.
*Animals from whole bone marrow transduction.

TABLE 3
Serial transplantation of AML1-ETO bone marrow

| Time post- | | | | | |
|---|---|---|---|---|---|
| transplant | 1° | 2° | GFP+ HSC | GFP+ HSC (Abs | GFP+ WBM |
| (mo) | recpt | recpt | (%) | #) | (%) |
| 2 | A | | 75.0 | 7,755 | 3.4 |
| 1 | | A1 | 0 | 0 | 0 |
| | | A2 | 0 | 0 | 0 |
| | | A3 | 97.2 | 347,976 | 1.8 |
| | | A4 | 0 | 0 | 0 |
| 10 | E | | 97.3 | 491,559 | 75.4 |
| 4 | | E1 | ND | ND | 35.0 |
| 4 | | E2 | ND | ND | 18.1 |
| 6 | | E3 | 69.8 | 5,641 | 11.8 |
| 2 | | E4 | 86.9 | 21,134 | 37.2 |

recpt = recipient. Secondary recipients each received 4 × 10⁶ whole bone marrow cells from their primary recipient.

TABLE 4

| | | | reconstituted | |
|---|---|---|---|---|
| retrovirus | tamoxifen | cell dose* | animals† | Chimerism |
| control | − | 200 | 0/4 | |
| | | 33 | 0/1 | |
| | | 7 | 0/3 | |
| control | + | 200 | 0/1 | |
| AML/ETO-ER | − | 200 | 0/3 | |
| | | 33 | 0/2 | |
| | | 7 | 0/3 | |
| AML/ETO-ER | + | 200 | 2/3 | 3.17% |
| | | 33 | 0/2 | |
| | | 7 | 0/2 | |

*Representative number of cells at initiation of culture
†Fraction of number of reconstituted animals to total number of transplanted animals for that dose

1. A method for expansion of a stem cell comprising contacting the stem cell in vitro with an amount of a modulator of an AML1-ETO target factor function effective to inhibit differentiation of the stem cell while not inhibiting self-renewal of the stem cell, and exposing the stem cell to cell growth conditions such that the cell proliferates.

2. The method of claim 1 wherein the cell is a hematopoietic stem cell.

3. The method of claim 2 where the hematopoietic stem cells are isolated from bone marrow, cord blood, peripheral blood CD34+ cell populations or peripheral blood CD34− cell populations.

4. The method of claim 2 where the hematopoietic stem cells are derived from a human.

5. The method of claim 2 where the expansion is carried out in vitro.

6. The method of claim 1 where the stem cells are isolated from a tissue selected from the group consisting of pancreas, muscle, nerve, skin and adipose.

7. Cancelled

8. The method of claim 6 where the expansion is carried out in vivo.

9. The method of claim 1 where the AML1-ETO target factor is a transcription factor.

10. The method of claim 9 where the transcription factor is selected from the group consisting of AML1, C/EBP alpha, PU.1 and a combination of any of the foregoing.

11. The method of claim 1 where the modulation of an AML1-ETO target factor function is an inhibition of function.

12. The method of claim 1 where the modulation of an AML1-ETO target factor function is a stimulation of function.

13. The method of claim 1 where the modulation of an AML1-ETO target factor function is a translocation of function.

14. The method of claim 11 where the inhibition occurs as a result of inhibition of the synthesis of the AML1-ETO target factor.

15. (canceled)

16. (canceled)

17. The method of claim 11 where the inhibition occurs as a result of inhibition of interaction with cellular factors that contributes to the activity of the AML1-ETO target factor.

18. (canceled)

19. (canceled)

20. The method of claim 11 where the inhibition occurs as a result of inhibition of DNA binding of the AML1-ETO target factor.

21. (canceled)

22. (canceled)

23. The method of claim 11 where the inhibition occurs as a result of stimulated degradation of mRNA encoding the AML1-ETO target factor.

24. (canceled)

25. (canceled)

26. The method of claim 11 where the inhibition occurs as a result of inhibition of transcription of the AML1-ETO target factor.

27. (canceled)

28. (canceled)

29. The method of claim 26 where said inhibition of transcription is accomplished using small interfering RNAs.

30. The method of claim 1 where the modulator of an AML1-ETO target factor function is an AML1-ETO fusion protein.

31. The method of claim 30 where said AML1-ETO fusion protein is expressed in said stem cell.

32. The method of claim 31 where said expression is a transient expression.

33. The method of claim 30 where said AML1-ETO fusion protein comprises a domain that reversibly activates and inactivates an AML1-ETO fusion protein function in the presence and absence, respectively, of an inducer.

34. The method of claim 33 where the domain is the hormone binding domain of the estrogen receptor and the inducer is estrogen or tamoxifen.

35. The method of claim 1 where the modulator of an AML1-ETO target factor function is an inhibitor of an AML1 activity.

36. The method of claim 1 where the modulator of an AML1-ETO target factor function is an inhibitor of a C/EBP alpha activity.

37. The method of claim 1 where the modulator of an AML1-ETO target factor function is an inhibitor of a PU.1 activity.

38. The method according to claim 1 wherein said contacting is carried out by culturing said precursor cell in medium containing a purified agonist in soluble form.

39. The method according to claim 1 wherein substantially no differentiation of the cell occurs.

40. The method of claim 1 where said modulating is direct or indirect.

41-123. (canceled)
